(19) United States
(12) Patent Application Publication
(10) Pub. No.: US 2023/0213439 A1
(43) Pub. Date: Jul. 6, 2023

(54) System and Method of Measuring Contaminants in a Substantially Translucent Material, Such as Water

(71) Applicant: Hypersoniq B.V., Capelle aan den IJssel (NL)

(72) Inventor: Lazlo Kleczewski, Oostzaan (NL)

(21) Appl. No.: 18/120,148

(22) Filed: Mar. 10, 2023

Related U.S. Application Data
(63) Continuation-in-part of application No. 17/220,053, filed on Apr. 1, 2021.

(30) Foreign Application Priority Data
Apr. 5, 2020 (EP) 20020155.6

(51) Int. Cl. G01N 21/33; G01N 33/18; G02B 6/10; G01N 21/35

(52) U.S. Cl. CPC G01N 21/33; G01N 33/18; G02B 6/102; G01N 21/35; G01N 2223/1003; G01N 2201/062; G01N 2201/06113

(57) ABSTRACT

A system for sensing analyte in at least partly translucent material, including one or more radiation sources configured for successively providing radiation at a first and a second wavelength, respectively, two or more waveguides for simultaneously transmitting the radiation at each wavelength provided by the radiation source, a first waveguide being a reference waveguide and a second being a sensing waveguide; and measuring means for measuring a phase difference between the radiation waves from the reference waveguide and the measuring waveguide, resp. The present method can be used for measuring contaminants such as Fe, Sn, and/or Pb in oil related products, such as carburant or lubricant.

### CROSS-REFERENCE TO RELATED APPLICATION

This application is a continuation-in-part of U.S. patent application Ser. No. 17/220,053, filed Apr. 1, 2021, which claims priority to European Patent Application No. 20020155.6, filed Apr. 5, 2020, the disclosures of which are hereby incorporated by reference in their entirety.

### BACKGROUND OF THE INVENTION

### Field of the Invention

This application relates to a system and method of measuring the quantity of analyte in at least partly translucent material.

### Description of Related Art

Interferometers (Mach-Zehnder, Michelson, Young etc.) are primarily used to measure phase differences (speed difference, refractive index) between two waves of light or other radiation for which purpose they show sufficient sensitivity even at the speed of light.

### SUMMARY OF THE INVENTION

The present invention provides a system for sensing analyte in at least partly translucent material, comprising:
one or more radiation sources configured for successively providing radiation at a first and a second wavelength, resp.;
two or more waveguides for simultaneously transmitting the radiation at each wavelength provided by the radiation source, a first waveguide being a reference waveguide and a second being a sensing waveguide; and
measuring means for measuring a phase difference between the radiation waves from the reference waveguide and the measuring waveguide, resp..

Preferably the different wavelength are provided by a number of monochromatic light sources which are switched ON/OFF one after the other.

The different wavelengths can preferably also be provided by a single (broad band) source, e.g. by using optical filtering and/or changing the temperature of the source.

The research of electrochemical reactions nowadays goes into the direction of measuring the dielectric properties of a medium as a function of frequency. It is an experimental method of characterizing electrochemical systems. This technique measures the impedance of a system over a range of frequencies. Often, data obtained by electrochemical impedance spectroscopy (EIS) are expressed graphically. The frequency ranges are each suited for different media. At high frequencies electronics may become a limiting factor.

The present invention also provides a method of measuring the quantity of analyte in at least partly translucent material, wherein the above system is used, and wherein both the absorption and the phase difference for different wavelengths one after the after is measured such that the composition and quantity of different analytes can be determined.

Preferably the radiation is in the high frequency optical range, viz. extending from near IR into UV, so that the measurements are very accurate for optically translucent materials, also called transparent materials.

The present preferred method can be used for measuring contaminants such as Fe, Sn, and/or Pb in oil related products, such as carburant or lubricant. Also the quality of pharmaceutical solutions and liquid food products, such as olive oil can be monitored. Preferably the method is used for monitoring contaminants in water; if phase difference values increase the operator can easily check the spectrum of the measured analytes.

### BRIEF DESCRIPTION OF THE DRAWINGS

Further details, advantages and characteristics of the present invention are clarified in the following description of preferred embodiments thereof referring to the annexed drawing, in which show:

FIG. 1 a schematic diagram of one preferred embodiment of the present invention;

FIG. 2 a schematic diagram of another preferred embodiment of the present invention;

FIG. 3 a schematic diagram of yet another preferred embodiment of the present invention; and

FIG. 4 a schematic diagram of still another preferred embodiment of the present invention.

### DESCRIPTION OF THE INVENTION

Various non-limiting examples will now be described with reference to the accompanying figures where like reference numbers correspond to like or functionally equivalent elements.

Substances showing transparent properties are most likely equal, if the refractive indexes are. Small changes to the material composition will typically result in subtle changes in the refractive index that can be measured.

Using an interferometer is a highly sensitive method that is able to detect very subtle changes in the refractive index.

For example water from different wells, will typically have slightly different refractive indexes that could both be perfectly safe for consumption. While a small trace amount of certain harmful chemicals dissolved in the water can cause similar changes in the refractive index, making it unsafe for consumption. It would be useful to give early warning of a possible problem, which allows for preventing harmful chemicals ending up in the drinking water supplied to users.

Once a change in the refractive index of the substance (water) would have been detected, additional testing could be done to identify the source of the change. This would imply taking a sample and analyse this sample in a lab.

Further sensors could also be used to search for contamination candidates.

This takes times also because each contaminant requires a dedicated sensor. A setup that is able to detect any contaminant is practically impossible.

A method that has both a high sensitivity and the ability to identify the chemicals is disclosed in the present patent application.

The refractive sensor, that is using monochromatic light, required for the internal interferometer, effectively gets two signals, the refractive index, and the light absorption at that particular wavelength (within a very narrow band of e.g., 10 nm). The refractive measurement uses a single wavelength at a time.

According to the present patent disclosure a number a light source are used that generate different wavelengths in sequence, or a wavelength sweep, so that it becomes possible to get more detailed information of the exact wavelengths with the amount of light absorption, and thus also obtain information about the chemical composition of the material tested by the refractive sensor, such as in gas chromatography in which method the measured spectrum is used to identify the composition of materials.

The present patent disclosure allows the high sensitivity of the refractive index measurements, and thus detecting very small changes while at the same time identifying the chemical compounds that are the cause of the change in the refractive index, and thus bypassing the need to do time consuming external testing to evaluate if corrective measures are to be taken.

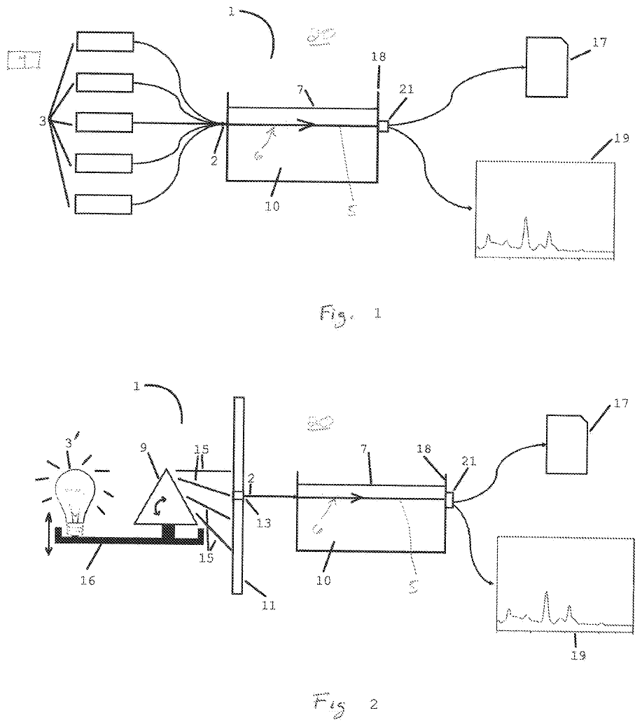

A light source that would make this possible could be for example in a system 1 (FIG. 1) a series of solid state lasers/LEDs 3 each tuned to a single wavelength and each connected to a voltage source 4. At connection point 2 the lasers/LEDs are connected to a pair of waveguides, of which a sensing waveguide extends through a substance 10, such as water with some salts or other chemical or physical impurities dissolved or emulsified. Herein, terms such as “substance 10”, “sample solution 10”, “sample 10”, and the like may be used interchangeably.

The sensing waveguide 5 extending from connection point 2 under the fluid surface 7 in a holder or container 18 through an end wall thereof into measuring unit 21 is e.g. provided with a window through which the refractive index (i.e. the velocity of light) is influenced by the amount of analyte present in the fluid. Herein, terms such as “measuring unit 21”, “light receiver 21”, “receiver 21”, and the like may be used interchangeably.

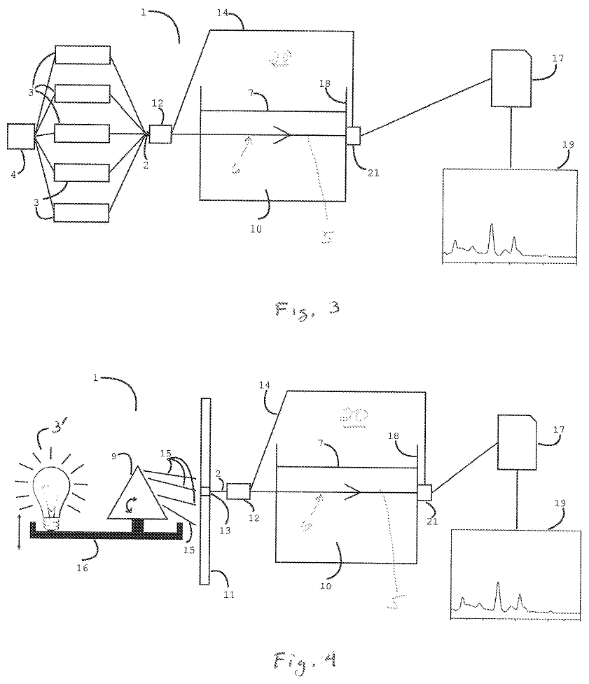

In measuring unit 21 the phase difference relative to the radiation in the reference waveguide 14 (shown in FIGS. 3 and 4) is measured by a number of photo diodes which detect the interference pattern between the two waves as well as the absorption of the waves at the particular wavelength.

It is apparent to the skilled artisan that the reference waveguide 14 has to be shielded from the environment, or has to be extending in a stable well known environment or reference environment such that calibration can be executed. In the figures, light travelling through the reference waveguide 14 is shielded from the environment via a top cladding on the material of the reference waveguide 14 wherein the light waves propagate.

By using etching techniques, the top cladding is locally removed at a well-defined position of the sensing waveguide 5 such as to provide a sensing window 6, the evanescent field of the light that travels through the sensing waveguide 5, extending into the environment above or around the sensing waveguide 5 and becoming susceptible to changes in refractive index of the sample solution 10 above or around of the sensing window 6.

The resulting change of the effective refractive index leads to a change of the speed of the light in the sensing waveguide 5 and a change in the relative phase between light that has travelled through the sensing waveguide 5 as compared to the reference waveguide 14. This change in relative phase leads to a change in the interference between light coming from the sensing and reference waveguides 5, 14 at a combining section, e.g., a measurement unit 21, and manifests itself as a change in the output intensity of the Mach-Zehder interferometer.

As schematically indicated at 19 a spectrum for analysing the composition of the analyte (contaminant) is provided as well as a very accurate measure 17 of the difference in refractive index related to the amount of the contaminant.

As soon as the phase difference starts changing an operator can see whether also the spectrum starts changing.

The Laser/LEDS are switched on/off in sequence such that only one is active at a given moment in time. It would also be possible to use the wavelength tuning mechanism of these light sources to sweep through a wavelength range.

Another method (FIG. 2) uses a single white light source (for example a halogen lamp) 3′ on a movable table 16, which light source shines through a rotating prism 9 to a slotted filter plate 11 showing opening 13 so that only a very narrow band of the light rays 15 from the prism is allowed to actually pass through. This will then create a light with the very narrow wavelength range that can be used by the interferometer of the refractive sensor.

By moving the light source, the prism or the slotted plate, or a combination thereof, it is possible to change the wavelength that is used by the sensor and thus sweeping through the wavelengths. The reference numerals of FIG. 1 are also used for the same or equivalent parts in FIG. 2.

In the examples of FIGS. 1 and 3, the source of the electromagnetic radiation is a group of different light sources 3 which are, for example, a series of solid-state Light Emitting Diodes (LEDs), each emitting at a different wavelength. Although the wavelength of the visible spectrum is in the region between 380 and 740 nanometres, as source(s) of radiation can be used electromagnetic emitters which emit in the ultraviolet or the infrared regions or even beyond them.

The electromagnetic sources 3 may be in parallel connection as represented in FIGS. 1 and 3 and they can be configured to be switched on and off in sequence by a driver 4, so that only one of them is active at a single moment of time and a single wavelength is generated.

Alternatively, one or different electromagnetic radiation sources 3 may be connected to a wavelength tuning mechanism, which is not shown. The wavelength tuning mechanism is able to make the light sources sweep in discrete steps through a predetermined range of wavelengths.

The radiation of a single wavelength is the input radiation beam 2, which passes through a splitter 12 (shown in FIG. 3) and is then transmitted through the sample 10 to a refractive sensor 21. The sample 10 to be examined is contained in a holder or container 18.

In different examples shown in FIGS. 2 and 4, the input of the apparatus comes from a single white light source 3′, for example a halogen lamp, a prism 9 and a slotted filter plate 11. The light source 3′ is in line with the prism 9 and between the prism 9 and the holder or container 18 containing the medium to be examined there is the slotted filter plate 11.

The prism 9 is a dispersive prism. Typical dispersive prisms are widely available in the market, they are transparent, and they are usually made of glass, plastic or fluorite.

The slotted filter plate 11 is not transparent and it includes a small opening 13. The dimension of the opening 13 is such to allow only a very narrow band of light to pass through.

The white light generated by the white light source 3′ is a combination of lights of different wavelengths in the spectrum. As the light source 3′ emits the white light, it passes through the prism 9 and it is broken up to its constituent spectral colors or wavelengths, for example the colors of the rainbow. According to the geometry of the prism 9, all the beams 15 of different wavelengths may be obtained when the prism is idle, or it may be necessary for the prism 9 to be rotated. Both the light source 3′ and the prism 9 are positioned or immovably fixed on a platform 16, which is able to move in a direction parallel to the slotted plate 11, as shown by the two-headed arrows in FIGS. 2 and 4. The movement of the platform 16 is made by a stepping or servo motor (not shown in the Figures), which assures a specific number of stops at precise intervals. At each stop of the platform 16, only a narrow beam 15, e.g., a few or a single wavelength, is able to pass through the opening 13 of the slotted filter plate 11, an optional splitter 12 (shown in FIG. 4), and through the sample 10 to be examined. This creates a light of a very narrow (or single) wavelength range which is then transmitted through the sample 10 to be examined and is received by the interferometer of the measuring unit 21, e.g., a refractive sensor.

Alternatively, the light of a very narrow wavelength range can be created by moving the slotted filter plate 11 in parallel with the holder or container 18 and the measuring unit 21.

The splitter 12 shown in FIG. 4 separates the radiation beam into two parts. The first part is crossing through the holder or container 18 containing the sample 10 via the sensing waveguide 5, whilst the other part is transmitted through the reference waveguide 14, such as an optical fiber, to the receiver 21 and constitutes the reference signal.

In the apparatus described above, instead of the slotted filter plate 11, a group of narrow band color filters or any other means that would allow the creation of a light with a very narrow wavelength band may be used.

The measuring unit or light receiver 21 is positioned at the side of the holder or container 18 opposite to the radiation source 3 or 3′ and is able to measure variations of the intensity of the light transmitted through each of the sensing waveguide 5 and the reference waveguide 14.

In parallel with the transmission of a sensing beam of light being transmitted through the sensing waveguide 5, a reference beam of light is transmitted through a reference waveguide (or optical fibre) 14 to the receiver 21, and serves for measuring the phase shift of the sensing beam of light which is crossing through the sample solution 10. With this method variations of the phase of the light crossing through the sample may be measured.

In the examples shown in FIGS. 3 and 4, the reference waveguide 14 extends outside the sample solution 10 in a reference environment (e.g., the ambient or external environment 20 surrounding the holder or container 18 containing the sample solution 10) of which the light conditions and (e.g., air or other environmental gas, e.g., one or more inert gasses) temperature are known and have a constant value, so that any phase shift of the reference beam of light propagating inside the reference waveguide 14 may be known a priori. It is apparent that there can also be a shielding around the reference waveguide 14 to shield the light inside thereof from the environmental light.

Alternatively, or additionally, the measuring unit 21 may include a CCD camera can be used to capture the spectrum of the light which is diffracted or scattered at small angles. The spectrum of the light diffracted at small angles may be analysed via mathematical inversions (e.g. Fourier transformation) and give essential information on the size of the diffracting particles.

By varying the wavelength of the light source input into the connection point 2 of the interferometer, the refractive index, the phase shift, the absorption and the diffraction of the light of every specific wavelength crossing the sample solution 10 can be measured. Because the absorption and/or transmission, the phase shift, the refractive index and the diffraction spectrum of light of specific wavelengths is related to the elements contained in the sample to be examined and the concentration of these elements in the sample, it is possible to identify the composition of the sample.

At a calibration phase of the process, a specific contaminant A in distilled water may be introduced into the holder or container 18 and the optical behaviour (absorption, phase shift, refractive index, diffraction spectrum) of the composition is measured at a number, e.g. eight, discrete wavelengths. These measured values may then be saved in a library. The concentration of the contaminant may then be doubled and the measurements repeated. The concentration of the contaminant may then be doubled again and the measurements repeated e.g., ten times. Thus, a library of the optical behavior of contaminant A in distilled water may be constituted.

The above steps may be repeated with a contaminant B and thus constitute a library of the optical behaviour of contaminant B in distilled water.

The same exercise may be repeated for a contaminant C and as many other contaminants as may be suspected of polluting water or other media.

The same exercise may be repeated for combinations of A+B, A+C, B+C etc. in such a manner that a complete library of values may be constituted

At the measuring phase of the process, the same values may be measured under the same conditions of temperature and pressure as the ones of the calibration phase.

At the comparison phase, the values obtained at the measuring phase are compared with the values in the library and the desired result is obtained.

The apparatus and method described above can be used for detecting the presence of undesired contaminants in water, air and other fluids. It can also be used for measuring the quantity or concentration of desired substances in a fluid or for any other application in which a non-soluble substance is suspended in an optically transparent fluid, such as air, oxygen, nitrogen, water, etc.

Other possibilities as not limiting examples, are the use of narrow band colour filters, and any other means that would allow the creation of a light with a very narrow wavelength band, whereby the base wavelength can be modified over a range.

The present invention is not limited to the description and embodiments above; the requested rights are determined by the following claims, within the scope of which many modifications are feasible.

1. A method of measuring the quantity of analyte in at least partly translucent material, wherein both an absorption and a phase difference for different wavelengths one after the other is measured such that a composition and quantity of different analytes can be determined, the method comprising:
successively providing light radiation, from one or more light radiation sources, at a first wavelength and a second wavelength;
simultaneous transmitting, by a first waveguide and a second waveguide, the light radiation at each wavelength provided from the one or more light radiation sources, wherein the first waveguide is a reference waveguide and the second waveguide is a sensing waveguide; and
measuring, by a measuring means, a phase difference between light radiation waves from the reference waveguide and the sensing waveguide after passage of the light radiation waves in a single direction through the reference waveguide and the sensing waveguide, wherein:
the sensing waveguide extends to the measuring means through a quantity of analyte in at least partly translucent material; and
the reference waveguide extends to the measuring means through a reference environment external to the quantity of analyte in the at least partly translucent material, wherein the reference environment does not include the analyte or another instance of the analyte or the at least partly translucent material or another instance of the at least partly translucent material.

2. The method of claim 1, further including switching each of a number of the one or more light radiation sources ON and OFF sequentially in time such that the light radiation is swept over different wavelengths.

3. The method of claim 2, wherein each of the number of the one or more light radiation sources comprises a monochromatic light source to provide (near) visible light at different wavelengths.

4. The method of claim 1, further including monitoring absorption of light radiation at different wavelengths when the quantity of analyte has changed.

5. The method of claim 1, wherein the translucent material is water, and the analyte is salt or any other chemical or physical water contaminant.

6. The method of claim 5, further including providing at least one of a spectrum related to a composition of the analyte or a measure of a difference in a refractive index between the sensing waveguide and the reference waveguide related to the amount of the contaminant.

7. The method of claim 1, wherein the light radiation source provides a range of different wavelengths, by a filter to transmit light radiation of one wavelength after the other to the waveguides.

8. The method of claim 1, wherein the one or more light radiation sources emit radiation in two or more wavelengths.

9. The method of claim 1, wherein the measuring means detects changes in a refractive index caused by absorption of the light radiation in the sensing waveguide.

10. The method of claim 2, wherein the different wavelengths extend from the infrared spectrum into the ultraviolet range.

11. The method of claim 1, wherein the measuring means comprise an array of photodetectors to establish a phase difference between the light radiation waves from the reference waveguide and the sensing waveguide.

12. The method of claim 3, wherein:
each monochromatic light source is a laser or an LED; and
the number of monochromatic light sources is between five and fifteen.

13. The method of claim 7, wherein the measuring means detects changes in a refractive index caused by absorption of the light radiation in the sensing waveguide.

14. The method of claim 7, wherein the different wavelengths extend from the infrared into the ultraviolet wavelengths.

15. The method of claim 7, wherein the measuring means comprise an array of photodetectors to establish a phase difference between the light radiation waves from the reference waveguide and the sensing waveguide.

16. The method of claim 1, wherein the translucent material is a liquid and the reference environment is an ambient environment.

17. The method of claim 16, wherein the ambient environment is air.
